(19) United States
(12) Patent Application Publication
(10) Pub. No.: US 2006/0240000 A1
(43) Pub. Date: Oct. 26, 2006

(54) Combination treatments for allergic disease comprising administering an anti-IgE antibody and antiallergic compound

(76) Inventors: Charles Edward Owen, Horsham (GB); Howard George Fox, Horsham (GB); Christoph Walker, Horsham (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/455,431

(22) Filed: Jun. 19, 2006

Related U.S. Application Data
(63) Continuation of application No. 10/500,200, filed on Oct. 8, 2004, now abandoned.

(30) Foreign Application Priority Data
Jan. 9, 2002 (GB) 0200429.9

(51) Int. Cl. A61K 39/395; A61K 31/4745

(52) U.S. Cl. 424/131.1; 514/183; 514/291

(57) ABSTRACT

Pharmaceutical composition comprising an anti-IgE antibody and at least one further antiallergic compound selected from the group consisting of anti-inflammatory agents, leukotriene modifiers, bronchodilators, antihistamines, interleukin antagonists, mast cell inhibitors, and immunotherapeutical agents, such as 33-epichloro,33-desoxyzascomycin (pimecrolimus), in which the active ingredients are present in each case in free form or in the form of a pharmaceutically acceptable salt and optionally at least one pharmaceutically acceptable carrier; for simultaneous, separate or sequential use.

### BACKGROUND OF THE INVENTION

The generally accepted aims in the treatment of allergic disease, are to provide relief from symptoms, improvement of the quality of life and prevention of both acute and chronic complications. Treatment of allergic disease varies with the severity and type of the symptoms. Short-term goals include relieving immediate symptoms, while long-term goals also include avoiding future allergic reactions. In order to achieve the therapeutical goals, it is often necessary to give medication to the patient having allergic disease. For example, corticosteroids such as dexamethasone or prednisone reduce the immune response and may be prescribed to reduce symptoms in allergic disease, antihistamines such as diphenhydramine may provide good relief of mild to moderate symptoms and epinephrine may be used to reduce swelling of the airways and other life-threatening symptoms of allergic disease. Generally, avoidance of the allergen is important for long-term treatment, particularly with allergic reaction to foods or medications. Also, desensitization (immunotherapy) is occasionally recommended if the allergen cannot be avoided. Desensitization includes regular injections of the allergen, given in increasing doses.

For example, in allergic asthma the treatment is aimed at controlling symptoms through medication. A variety of medications for treatment of allergic asthma are available. These medications include antiallergic compounds of various chemical and therapeutical classes, such as, for example, anti-inflammatory substances, leukotriene inhibitors, bronchodilators, cromolyn sodium and amino- or theophylline. Patients with mild asthma, i.e. having infrequent attacks, may use bronchodilators as needed while those with significant asthma, e.g. symptoms occurring more than twice per week, should be treated with anti-inflammatory medications, preferably inhaled corticosteroids, and with inhaled bronchodilators in addition. Acute severe asthma requires a medical evaluation and may require hospitalization, oxygen, and intravenous medications.

However, there generally remains a need to improve the presently available medication in order to better control the symptoms and to ameliorate the underlying disease processes in order to meet the therapeutic challenge of controlling allergic disease.

### SUMMARY OF THE INVENTION

The present invention provides a pharmaceutical composition comprising an anti-IgE antibody and at least one further antiallergic compound selected from the group consisting of anti-inflammatory agents, leukotriene modifiers, bronchodilators, antihistamines, interleukin antagonists, mast cell inhibitors, and immunotherapeutical agents, such as 33-epichloro,33-desoxyascomycin (pimecrolimus), in which the active ingredients are present in free form or in the form of a pharmaceutically acceptable salt and optionally at least one pharmaceutically acceptable carrier; for simultaneous, separate or sequential use.

Also provided is a method for the prevention, delay of progression or treatment of allergic disease comprising administering to a warm-blooded animal a therapeutically effective amount of the composition of the invention.

Furthermore, a method of treatment of allergic disease is provided comprising administering to a warm-blooded animal a therapeutically effective amount of an anti-IgE antibody and an antiallergic compound.

In another aspect of the invention there is provided the use of the composition of the invention in medicine.

Furthermore, the use of a composition according to the invention for the manufacture of a medicament for the treatment of a warm-blooded animal having allergic disease is provided.

In another aspect, there is provided a kit comprising as active agent a composition according to this invention together with instructions for simultaneous, separate or sequential use thereof in the prevention, delay of progression or treatment of allergic disease.

### DETAILED DESCRIPTION OF THE INVENTION

The present invention relates to a combination, such as a combined preparation or pharmaceutical composition, respectively, which comprises an anti-IgE antibody and at least one further antiallergic compound selected from the group consisting of anti-inflammatory agents, leukotriene modifiers, bronchodilators, antihistamines, interleukin antagonists, mast cell inhibitors, and immunotherapeutical agents, in which the active ingredients are present in free form or in the form of a pharmaceutically acceptable salt and optionally at least one pharmaceutically acceptable carrier; for simultaneous, separate or sequential use, especially in the prevention, delay of progression or treatment of allergic disease, especially allergic asthma, seasonal allergic rhinitis, perennial allergic rhinitis and atopic dermatitis, and diseases and conditions associated with allergic disease.

The anti-IgE antibody and at least one further antiallergic compound as mentioned above can be dosed independently or by use of different fixed combinations with distinguished amounts of the components. The parts of the kit of parts can then e.g. be administered chronologically staggered, that is at different time points and with equal or different time intervals for any part of the kit of parts. Preferably, the time intervals are chosen such that the effect on the treated disease or condition in the combined use of the parts is larger than the effect which would be obtained by use of only any one of the components. Preferably, there is at least one beneficial effect, e.g. a mutual enhancing of the effect of the active ingredients, additional advantageous effects, less side-effects, a combined therapeutical effect in a non-effective dosage of one or each of the active ingredients, and especially a synergism, e.g. a more than additive effect, between an anti-IgE antibody, respectively, and the at least one further compound as mentioned above.

An “anti-IgE antibody” within the meaning of the invention may be any antibody directed against an IgE antibody, in particular an antibody directed against the Fc portion of an IgE antibody. Preferably the anti-IgE antibody is a humanized murine antibody or a fully human antibody. Preferably the anti-IgE antibody is a non-anaphylactogenic anti-IgE antibody. Thus, preferably, the IgE antibodies of the instant invention do not result in histamine release from mast cells or basophils.

Preferred anti-IgE antibodies of the invention are the antibodies named Omalizumab (E25), E26, CGP56901, CGP51901, or their fragments and derivatives, as further defined hereinbelow. Most preferably the anti-IgE antibody is Omalizumab, which is also named “E25”. Another particularly preferred anti-IgE antibody is named “E26” as further defined hereinbelow.

Generally, anti-IgE antibodies are described in the prior art, and in greater detail in the International applications WO 93/04173 and WO 99/01556. For example, WO 99/01556 specifically describes Omalizumab, in FIG. 12, and in the sequences ID-No. 13-14. Antibody molecules comprising a E26 sequence are described in WO 99/01556 and are selected from the group of F(ab) fragment (Sequence ID Nos. 19-20), sFv fragment (Sequence ID No. 22) and F(ab)′₂ fragment (Sequence Nos. 24-25), in accordance to FIGS. 12-15. Within this invention, the terms E25 and E26 shall be construed accordingly.

Also included in the present invention are the antibodies as specifically described in US patents U.S. Pat. No. 6,066,718; U.S. Pat. No. 6,072,035 and U.S. Pat. No. 5,958,708.

U.S. Pat. No. 5,449,760 generally describes anti-IgE antibodies that bind soluble IgE but not IgE on the surface of B cells or basophils. Antibodies such as these bind to soluble IgE and inhibit IgE activity by, for example, blocking the IgE receptor binding site, by blocking the antigen binding site and/or by simply removing the IgE from circulation. Additional anti-IgE antibodies and IgE-binding fragments derived from the anti-IgE antibodies are described in U.S. Pat. No. 5,656,273. U.S. Pat. No. 5,543,144 describes anti-IgE antibodies that bind soluble IgE and membrane-bound IgE on IgE-expressing B cells but not to IgE bound to basophils.

The “antiallergic compound ” of the invention may be selected from the group consisting of (1) anti-inflammatory agents, (2) leukotriene modifiers, (3) bronchodilators, (4) antihistamines, (5) interleukin antagonists, (6) mast cell inhibitors, and (7) immunotherapeutical agents.

The term “anti-inflammatory agents” as used herein includes steroidal and non-steroidal agents useful for treating allergic disease. Preferred anti-inflammatory agents of the invention are corticosteroids and in particular “inhaled corticosteroids”. In particular, anti-inflammatory agents within the meaning of the invention may be selected from

inhaled corticosteroids, such as beclomethasone, flunisolide, triamcinolone, budesonide, fluticasone (Flovent™), dexamethasone;

intravenous corticosteroids, such as prednisone, methylprednisolone, hydrocortisone.

Other useful anti-inflammatory drugs are those with other antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly antagonists to the receptors CCR2, CCR3, CCR4 and CCR8. Examples are Schering-Plough antagonists SC-351125, SCH-55700 and SCH-D, Takeda antagonists such as N-[[4-[[[6,7-dihydro-2-(4-methylphenyl)-5H-benzocyclohepten-8-yl]crbonyl]amino]phenyl]-methyl]tetrahydro-N,N-dimethyl-2H-pyran4-aminium chloride (TAK-770), and CCR-5 antagonists described in U.S. Pat. No. 6,166,037 (particularly claims 18 and 19), WO0/66558 (particularly claim 8), and WO00/66559 (particularly claim 9). Also included are Phosphodiesterase (PDE4) inhibitors such as Cilomilast (Ariflo™), Roflumilast (Byk Gulden), V-11294A (Napp), BAY19-8004 (Bayer), SCH-351591 (Schering-Plough), and PD189659 (Parke-Davis).

The term “leukotriene modifiers” as used herein includes preferredly antagonists of leukotriene function. Such compounds may target the receptors involved in relevant leukotriene pathways or other enzymes involved in production or clearance of leukotrienes. In particular, leukotriene modifiers within the meaning of the invention may be selected from

the class of LTD4 receptor agonists, such as Zafirlukast (Accolate™) or Montelukast (Singulair™)

the class of 5-Lipoxygenase Inhibitors, such as Zileuton (Zyflo™)

the class of LTB4 antagonists such as LTB4 antagonists described in U.S. Pat. No. 5,451,700.

The term “bronchodilators” as used herein includes either short-acting (lasting a short time) or long-acting (lasting a long time) bronchodilators. They include for example anticholinergic or antimuscarinic agents, such as ipratropium bromide (Atrovent™), oxitropium bromide, tiotropium bromide, aminophylline, oxtriphylline, theophylline (Aerolate™), and, preferredly, beta2 adrenergic agonists.

“Beta2 adrenergic agonists” within the meaning of the invention may be selected from

long acting beta2 agonists, such as formoterol or salmeterol

short acting beta2 agonists, such as albuterol, bitolterol, epinephrine, fenoterol, isoetharine, isoproterenol, rrietaproterenol, pirbuterol, procaterol and terbutaline.

The term “antihistamines” as used herein includes compounds that reduce histamine release as well as compounds that block histamine function. Preferred combination partners are nonsedating antihistamines. In particular, antihistamines within the meaning of the invention may be selected from, loratidine (Claritin™), desloratidine (NeoClaritin™), cetirizine (Zyrtec™), levocetirizine (Xyzal™), astemizole (Hismanal™), norastemizole , acetaminophen, clemastine, promethazine, diphenhydramine and fexofenadine.

The term “interleukin antagonists” as used herein includes neutralising monoclonal antibodies directed against targets which include IL-4, IL-5, IL-8, IL-9, IL-13, ICAM-1, IL-17, IL-1 and its variants, MCP-1 and eotaxin. In particular, Interleukin antagonists within the meaning of the invention may be selected from ABX-IL8 (anti-lL8 antibody), SB 240683 (anti-IL4 antibody), SB 203580 (anti-IL1 antibody), Mepolizumab (anti-IL-5 antibody) and SCH 55700 (anti-IL5 antibody).

Furthermore, soluble receptor antagonists and/or fusion proteins are included within the group of interleukin antagonists. Preferred examples are in particular soluble receptor antagonists and/or fusion proteins directed against IL-4, IL-13 and IL-17. Also, the so-called mutein antibodies including BAY 169996 directed against IL-4 from Bayer are interleukin antagonists within the meaning of the invention. Thus, interleukin antagonists within the meaning of the invention may also be selected from soluble IL-4 receptor (Nuvance™), Interleukin-13 IgGFc fusion protein and soluble IL-1 receptor types I & II.

“Mast cell inhibitors” within the meaning of the invention may be selected from cromolyn medications (cromoglicic acid, cromoglycic acid, sodium cromoglicate , and sodium cromoglycate) (Intal™), Nedocromil (Tilade™), and Azelastine (Astelin™, Optivar™).

The term “immunotherapeutical agents” as used herein includes standard immunotherapy approaches known in the art, but also immune deviation approaches such as treatment with immunomodulators. In particular, immunotherapeutical agents within the meaning of the invention may be selected from Immunogenic peptides (eg. CatPAD (Heska), DNA vaccines (eg. Dynavax Amb a 1 immunostimulatory oligodesoxynibonucleotide conjugate (AIC)); CpG nucleotides) and SRL172 (SR Pharma).

A suitable immunomodulator is for example a macrolide T-cell immunomodulator or immunosuppressant. A macrolide T-cell immunomodulator or immunosuppressant is to be understood herein as being a T-cell immunomodulator or T-cell immunosuppressant which has a macrocyclic compound structure including a lactone or lactam moiety. While it preferably has at least some T-cell immunomodulating or immunosuppressant activity, it may also exhibit concomitantly or predominantly further pharmaceutical properties, such as anti-inflammatory activity. A preferred example of a macrolide T-cell immunomodulator or immunosuppressant is a FKBP12-binding calcineurin inhibitor or mitogen-activated kinase modulator or inhibitor, in particular an asco- or rapamycin. It preferably is an ascomycin. While the macrolide preferably has at least some calcineurin- or mitogen-activated kinase modulating or inhibiting activity, it may also exhibit concomitantly or predominantly further pharmaceutical properties, such as antiinflammatory activity. It preferably is a compound, e.g. an ascomycin, having rather long-acting activity relatively to other members of the same structural class, e.g. it is degraded metabolically slowly to inactive products. An asco- or rapamycin is to be understood as asco or rapamycin as such, or a derivative thereof. A derivative is to be understood as being an antagonist, agonist or analogue of the parent compound which retains the basic structure and modulates at least one of the biological, for example immunological properties of the parent compound.

Suitable ascomycins are e.g. as described in EP 184162, EP 315978, EP 323042, EP 423714, EP 427680, EP 465426, EP 474126, WO 91/13889, WO 91/19495, EP 484936, EP 523088, EP 532089, EP 569337, EP 626385, WO 93/5059 and WO 97/8182; in particular:

ascomycin;

tacrolimus (FK506; Prograf^{R});

imidazolylmethoxyascomycin (WO 97/8182 in Example 1 and as compound of formula I);

32-O-(1 -hydroxyethylindol-5-yl)ascomycin (L-732531) (Transplantation 65 [1998] 10-18, 18-26, on page 11, FIG. 1; and

(32-desoxy,32-epi-N1-tetrazolyl)ascomycin (ABT-281) (J. Invest. Dermatol. 12 [1999] 729-738, on page 730, FIG. 1);

preferably:

{1R,5Z,9S,12S-[1E-(1R,3R,4R)],13R,14S,17R,18E,21S,23S,24R,25S,27R}-17-ethyl-1,14-dihydroxy-12-[2-(4-hydroxy-3-methoxycyclohexyl)-1-methylvinyl]-23,25-dimethoxy-13,19,21,27-tetramethyl-11,28-dioxa-4-azatricyclo[22.3.1.0(4,9)]octacos-5,18-diene-2,3,10,16-tetraone (Example 8 in EP 626385), hereinafter referred to as “5,6-dehydroascomycin”;

{E-(1R,3R,4R)]1R,4S,5R,6S,9R,10E,13S,15S,16R,17S,19S,20S}-9-ethyl-6,16,20-trihydroxy-4[2-(4-hydroxy-3-methoxycyclohexyl)-1-methylvinyl]-15,17-dimethoxy-5,11,13,19-tetramethyl-3-oxa-22-azatricyclo[18.6.1.0(1,22)]heptacos-10-ene-2,8,21,27-tetraone (Examples 6d and 71 in EP 569337),

hereinafter referred to as “ASD 732”; and

pimecrolimus (INN recommended) (ASM981; Elidel™), i.e{[IE-(1R,3R,4S)]1R,9S,12S, 13R,14S,17R,18E, 21S,23S,24R,25S,27R}-12-[2-(4-chloro-3-methoxycyclohexyl)-1-methylvinyl]-17-ethy-1,14-dihydroxy-23,25-dimethoxy-13,19,21,27-tetramethyl-11,28,dioxa-4-azatricyclo [22.3.1.0(4,9)]octacos-18-ene-2,3,10,16-tetraone, of formula I

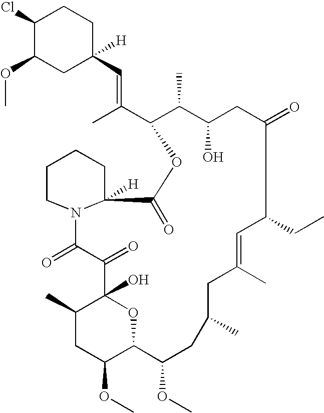

(Example 66a in EP 427680),

hereinafter referred to as “33-epichloro,33-desoxyascomycin”.

Suitable rapamycins are e.g. as described in U.S. Pat. No. 3,929,992, WO 94/9010 and U.S. Pat. No. 5,258,389, preferably sirolimus (rapamycin; Rapamune^{R}) and everolimus (RAD001; Certican^{R}).

One example of a suitable immunomodulator is given in Example 2. Thus, preferred embodiments.of the present invention include a macrolide T-cell immunomodulator or immunosuppressant. In one embodiment, the macrolide T-cell immunomodulator or immunosuppressant is a FKBP12-binding calcineurin inhibitor or mitogen-activated kinase modulator or inhibitor, in particular an asco- or rapamycin. Preferably it is an ascomycin, such as 33-epichloro,33-desoxyascomycin (pimecrolimus).

The structure of the active agents identified by code nos., generic or trade names may be taken from the actual edition of the standard compendium “The Merck Index” or the “Physician's Desk Reference (PDR)” or from databases, e.g. Patents International (e.g. IMS World Publications). The corresponding content thereof is hereby incorporated by reference. A person skilled in the art is fully enabled to identify the active agents and, based on these references, likewise enabled to manufacture and test the pharmaceutical indications and properties in standard test models, both in vitro and in vivo.

It will be understood that in the discussion of methods, references to the active ingredients are meant to also include the pharmaceutically acceptable salts. If these active ingredients have, for example, at least one basic center, they can form acid addition salts. Corresponding acid addition salts can also be formed having, if desired, an additionally present basic center. The active ingredients having an acid group (for example COOH) can also form salts with bases. The active ingredient or a pharmaceutically acceptable salt thereof may also be used in form of a hydrate or include other solvents used for crystallization.

The combination which comprises an anti-IgE antibody such as, for example, Omalizumab and at least one further compound selected from the group consisting of anti-inflammatory agents, leukotriene modifiers, bronchodilators, antihistamines, interleukin antagonists, mast cell inhibitors, and immunotherapeutical agents, in which the active ingredients, such as 33-epichloro,33-desoxyascomycin (pimecrolimus), are present in free form or in the form of a pharmaceutically acceptable salt, if at least one salt-forming group is present, will be referred to hereinafter as a COMBINATION OF THE INVENTION.

The combinations of the invention are appropriate for prevention of an allergic response as well as treating a pre-existing allergic condition.

The term “treatment” as used herein includes alleviation of one or more symptoms of the disorder, diminishment of the extent of the disorder, stabilization of the disorder, delay or slowing of disorder progression, amelioration or palliation of the disorder, and partial or total remission.

The terms “warm-blooded animal” or “mammal” include a human being.

The term “prevention” means prophylactic administration of the combination to healthy patients to prevent the outbreak of the diseases and conditions mentioned herein. Moreover, the term “prevention” means prophylactic administration of such combination to patients being in a pre-stage of the allergic disease to be treated.

The term “delay of progression” used herein means administration of the combination to patients being in a pre-stage of the allergic disease to be treated in which patients a pre-form of the corresponding disease is diagnosed.

The term “allergic disease” is to be understood according to its meaning in the art of medicine. In particular, allergic disease within the meaning of the invention includes a disease that is characterized by an allergic and/or atopic immunological reaction to an antigen, which results in allergic and/or atopic symptoms in the patient suffering from allergic disease. The term “allergic disease” in particular includes a disease which is characterized by elevated circulating IgE levels. An allergic disease often is characterized by the generation of antigen-specific IgE and the resultant effects of the IgE antibodies. As is well-known in the art, IgE binds to IgE receptors on mast cells and basophils. Upon later exposure to the antigen recognized by the IgE, the antigen cross-links the IgE on the mast cells and basophils causing degranulation of these cells.

Preferred examples of allergic disease are allergic asthma, allergic rhinitis, such as seasonal allergic rhinitis and perennial allergic rhinitis, and atopic dermatitis.

Allergic asthma as a clinical disorder that is characterized by airway inflammation; airway obstruction, which is reversible; and increased sensitivity, referred to as hyperreactivity. Obstruction to airflow is measured by a decrement in forced expired volume in one second (FEV I) which is obtained by comparison to baseline spirometry. Hyperreactivity of the airways is recognized by decreases in FEVI in response to very low levels of histamine or methacholine. Hyperreactivity may be exacerbated by exposure of the airways to allergen. Allergy testing can be helpful in identifying allergens in patients with persistent asthma. Common allergens include pet dander, dust mites, cockroach allergens, molds, and pollens. Common respiratory irritants include tobacco smoke, pollution, and fumes from burning wood or gas.

Allergic rhinitis is a clinical disorder characterized by nasal congestion, rhinorrhea, sneezing, and itching. Severity of these symptoms can vary from year to year, with occasional spontaneous remissions. Therefore, allergic rhinitis is classified by whether symptoms occur during certain seasons (SAR or seasonal allergic rhinitis) or year-round (PAR or perennial allergic rhinitis). The seasonal variety is usually caused by pollens from plants that depend on the wind for cross-pollination, such as grasses, trees, weeds, and mold spores. Serious complications, such as nasal polyps, recurrent sinusitis, recurrent ear infections, and hearing loss, can occur if allergic rhinitis is not treated or is undertreated. Psychosocial effects can include frequent absences from work or school, poor performance, poor appetite, malaise, and chronic fatigue.

Atopic dermatitis is a skin disorder involving hypersensitivity reaction within the skin characterized by inflammation, itching, and scaling. Atopic dermatitis can occur in an infantile or adult form. There is often a family history of asthma, hay fever, eczema, psoriasis, or other allergic diseases or allergy-related disorders. In adults, it is generally a chronic condition. Neurodermatitis is also a form of atopic dermatitis. It is characterized by a self-perpetuating scratch-itch cycle. Although symptoms increase in times of stress, physiological changes in the nerve fibers are also present. A hypersensitivity reaction occurs in the skin, causing chronic inflammation.

The nature of allergic disease and related diseases or conditions is multifactorial. Under certain circumstances, drugs with different mechanisms of action may be combined. However, just considering any combination of drugs having different mode of action but acting in the similar field does not necessarily lead to combinations with advantageous effects.

All the more surprising is the finding that the combined administration of a COMBINATION OF THE INVENTION, results in a beneficial, especially a synergistic, therapeutic effect and/or in additional benefits resulting from combined treatment such as a surprising prolongation of efficacy, a broader variety of therapeutic treatment and surprising beneficial effects on diseases and conditions associated with allergic asthma or seasonal allergic rhinitis compared to a monotherapy applying only one of the pharmaceutically active ingredients used in the COMBINATION OF THE INVENTION.

Accordingly, the invention provides a pharmaceutical composition which comprises an anti-IgE antibody such as, for example, Omalizumab and at least one further compound selected from the group consisting-of anti-inflammatory agents, leukotriene modifiers, bronchodilators, antihistamines, interleukin antagonists, mast cell inhibitors, and immunotherapeutical agents, or the pharmaceutically acceptable salts of such compounds where possible.

Preferably, the at least one further pharmaceutically active compound selected from the group above is a monoclonal antibody, such as and anti-interleukin antibody or a immunotherapeutical agent. Antibodies and vaccines are particularly suitable to be administered in a fixed combination for parenteral, and, in particular, subcutaneous administration.

A particularly preferred pharmaceutical composition comprises a combination of an anti-IgE antibody such as, for example, Omalizumab and a macrolide T-cell immunomodulator or immunosuppressant, such as, for example 33-epichloro,33-desoxyascomycin (pimecrolimus).

Treatment with Omalizumab, respectively, can commence prior to, subsequent to or concurrent with commencement of treatment with the Antiallergic Compound of the invention.

It will be understood that any statistically significant attenuation in the disease symptoms of allergic disease, such as allergic asthma or seasonal allergic rhinitis pursuant to the treatment of the present invention is within the scope of the invention.

In practical use, the antiallergic compounds or combinations thereof can be combined as the active ingredients in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed or carriers, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed.

The term “administering” also encompasses the use of prodrugs of any of the anti-allergic drugs that convert in vivo to the selective anti-allergic drug. The instant invention is therefore to be understood as embracing all such regimes of simultaneous or alternating treatment and the term “administering” is to be interpreted accordingly.

A further aspect of the present invention is the use of a pharmaceutical composition comprising the COMBINATION OF THE INVENTION for the preparation of a medicament for the prevention, delay of progression or treatment of allergic disease, in particular of allergic asthma or seasonal allergic rhinitis or a disease or condition associated with allergic disease.

There is further provided a method of prevention, delay of progression or treatment of and a pharmaceutical composition for the prevention, delay of progression or treatment of allergic disease. The treatment involves administering to a patient in need of such treatment a pharmaceutical composition comprising a pharmaceutical carrier and a therapeutically effective amount of the COMBINATION OF THE INVENTION.

A further aspect of the present invention is a method of treatment of a warm-blooded animal, especially a human, having allergic disease, in particular allergic asthma (AA), seasonal allergic rhinitis (SAR), perennial allergic rhinitis (PAR) and atopic dermatitis (AD) or a disease or condition associated with allergic disease, comprising administering to the animal a COMBINATION OF THE INVENTION in an amount which is jointly therapeutically effective against allergic disease in which the active ingredients can also be present in the form of their pharmaceutically acceptable salts simultaneously or sequentially in any order, separately or in a fixed combination.

The invention relates in particular to a kit or commercial package comprising jointly therapeutically effective amounts of COMBINATION OF THE INVENTION together with instructions for use thereof in the treatment of allergic disease, in particular allergic asthma (AA), seasonal allergic rhinibs (SAR), perennial allergic rhinitis (PAR) and atopic dermatitis (AD), or a disease or condition associated with allergic disease.

The therapeutically effective dosage of each of the active ingredients employed in the combination therapy may vary depending on the particular compound or pharmaceutical composition employed, the mode of administration, the condition being treated, the severity of the condition being treated, the species of the warm-blooded animal, body weight, sex, diet and age. Thus, the dosage regimen utilizing the compounds of the present invention is selected in accordance with a variety of factors including the route of administration and the renal and hepatic function of the patient. A physician, clinician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the drug's availability to target sites. This involves a consideration of the distribution, equilibrium, and elimination of a drug. Hence, the dosage regimen, i.e. dose level and frequency of dosage, of any of the individual components of the COMBINATION OF THE INVENTION as described hereinafter may be adjusted to provide the optimal therapeutic response.

In general, synergistically effective amounts of Omalizumab and 33-epichloro,33-desoxyascomycin (pimecrolimus) on administration for use in treatment of allergic disease, in particular allergic asthma (M), seasonal allergic rhinitis (SAR), perennial allergic rhinitis (PAR) and atopic dermatitis (AD), or a disease or condition associated with allergic disease, are, for example, amounts of Omalizumab according to Table 3, in combination or co-administration with amounts of 33-epichloro,33-desoxyascomycin (pimecrolimus) of up to about 2 mg/kg/day, e.g. from about 0.01 mg/kg/day to about 2 mg/kg/day, preferably about 0.5 mg/kg/day. Suitable unit dosage forms for co-administration of these compounds thus may, for example, contain, on the order of 125 mg to about 375 mg of Omalizumab and on the order of from about 0.5 mg to about 100 mg, preferably about 3 mg to about 30 mg of 33-epichloro,33-desoxyascomycin (pimecrolimus). The daily dosage for administration may be taken in a single dose, but may be spread out over two, three or four dosages. For systemic administration such as subcutaneous (s.c.) or intravenous (i.v), the effective dosage is lower than that required for oral administration, e.g. it may be about one fifth the oral dosage.

By “co-administration” is meant administration of the components of the compositions of the invention together, either in the same vehicle or in separate vehicles. The compounds may be administered as a fixed combination or may be administered in separate dosage forms. The compounds may also be administered at substantially the same time, e.g. within fifteen minutes or less, either in the same vehicle or in separate vehicles. Alternatively, Omalizumab may be administered before administering 33-epichloro,33-desoxyascomycin (pimecrolimus), e.g. four or two weeks, or only three days, or as little as about 15 min before receiving 33-epichloro,33-desoxyascomycin (pimecrolimus). Patients already under treatment with Omalizumab may receive 33-epichloro,33-desoxyascomycin (pimecrolimus) at any time within the treatment intervals of Omalizumab, such as the two weeks or four weeks intervals as described in Example 1 and Table 3, for example.

It will be appreciated that the unit content of active ingredient or ingredients contained in an individual dose of each dosage form need not in itself constitute an effective amount since the necessary effective amount can be reached by administration of a plurality of dosage units.

The invention will be more fully understood by reference to the following examples. They should not, however, be construed as limiting the scope of the invention.

### EXAMPLES

It can be shown by established test models and especially those test models described herein that the COMBINATION OF THE INVENTION results in a more effective prevention or, preferably, treatment of allergic disease, and in particular allergic asthma, seasonal allergic rhinitis, perennial allergic rhinitis and atopic dermatitis and diseases and conditions associated with allergic disease.

The person skilled in the pertinent art is fully enabled to select a relevant in vitro or animal test model to assess the hereinbefore and hereinafter indicated therapeutic indications and beneficial effects. Often the person skilled in the pertinent art will conduct a relevant clinical study to assess the hereinbefore and hereinafter indicated therapeutic indications and beneficial effects. For example, the anti-IgE antibody of the composition or combination to be tested may not cross-react with IgE from small mammals, other than primates and man.

### Example 1

Clinical Studies

Clinical studies prove, e.g., the synergism of the COMBINATION OF THE INVENTION. The beneficial effects on allergic disease and conditions associated with allergic disease as defined in this application can be determined directly through the results of these studies or by changes in the study designs which are known as such to a person skilled in the art.

The studies are, in particular, suitable to assess the effects of monotherapy with an anti-IgE antibody such as, for example, Omalizumab, and the other active ingredients mentioned herein in comparison to a COMBINATION OF THE INVENTION on for example, exacertion rates, symptom control, concomitant medication in use or other relevant functional parameters of allergic disease, such as for example the lung function. Measurement of free IgE may also serve as a marker of therapeutical efficacy. The length of the respective study depends on the combination to be tested, in many cases a duration of at least 16 weeks is likely to be needed.

Example for a Clinical Double-Blind, Randomized, Parallel-Group Studies in Subjects With Allergic Asthma to Assess Efficacy of an an Anti-IgE Antibody Administered Together With a Combination Partner

Subjects with a diagnosis of allergic asthma are chosen for these trials. The effects on the reduction of rescue medication intake (such as antihistamines and corticosteroids) and/or reduction of clinical symptoms are determined in this studies with the control achieved on placebo.

Efficacy parameter scores are mean and median daily symptom scores which are calculated based on the patient's diary assessment of clinical symptoms. Symptoms are categorized into 7 domains (stuffy nose, runny nose, itchy nose, sneezing and itchy eyes, watery eyes, red eyes). Each category can score 0-3 (none-mild-moderate-severe). Daily rescue medication scores are given: 0 for no medication; 1 for topical antihistamines; 2 for systemic antihistamines, 3 for oral or topical corticosteroids. Only maximal score per day is assessed.

The primary outcome variable is the symptom load (mean daily symptom score plus mean daily rescue medication score). Secondary clinical efficacy variables measured are: symptom score (mean of the daily symptom score), rescue medication score (mean of the daily rescue medication score during entire pollen season), proportion of days with rescue and/or concomitant medication use, investigator's global evaluation of treatment tolerability. Safety assessments include monitoring and recording of all adverse events and serious adverse events, hermatological, serum chemistry and urinary laboratory evaluations.

Before starting with the double-blind treatment for 16-24 weeks, the subjects are administered for 4 weeks an anti-IgE antibody such as, for example, Omalizumab, matching placebos and a placebo matching the combination partner (period I). The subjects are then separated into four treatment groups for the 16-24 weeks double-blind study (period II) as depicted in Table 1. Approximately 50 to 250 subjects are randomized per treatment group. The total study duration including the run-in period for each subject can be, e.g 20-28 weeks. Statistical analysis can be carried out by methods known in the art.

TABLE 1
Treatment groups for the double-blind study

1.) anti-IgE antibody* + combination partner placebo
2.) anti-IgE antibody placebo* + combination partner
3.) anti-IgE antibody* + combination partner
4.) anti-IgE antibody placebo* + combination partner placebo
*administered subcutaneously

In One Clinical Study Where the Anti-IgE Antibody is Omalizumab, the Following Administration and Dosage Scheme is Chosen:

Omalizumab is supplied as a sterile, freeze dried preparation that can be reconstituted to a final Omalizumab concentration of 125 mg/ml. Each 10 ml vial contains 208 mg rhuMAb-E25. Omalizumab must be stored refrigerated at (2°-8° C.) until time of administration to the subject, do not freeze. Each vial is reconstituted with 1.3 ml of Sterile Water for Injection (SWI), and the contents are gently swirled for 30 seconds, then left for up to 5 minutes to solubilize. 1.2 ml is then drawn up to deliver 150 mg of rhuMAb-E25. The formulation does not contain a preservative and is to be used for single-dose administration only.

After reconstitution, patients randomized to Omalizumab receive blinded test drug dependent on baseline IgE levels. The corresponding placebo group receive placebo dependent on IgE levels.

Omalizumab is administered using a disposable 25 gauge needle and a disposable plastic tuberculin-type syringe. The injections are administered in the deltoid region on the right arm. Alternately, the injections can be administered in the right thigh if medically significant reasons preclude administration in the deltoid region. The injections are administered subcutaneously.

The dose of Omalizumab which is based on baseline free serum IgE levels, is designed to suppresses free serum IgE to levels below 25 ng/ml. For example, in patients with asthma, Omalizumab 150-375 mg may be administered subcutaneously every 2 or 4 weeks (see dosing scheme of Tables 2 and 3).

TABLE 2
Omalizumab Dosing Schedule/Number of injections per dose (mg)

| Dose (mg) | Number of injections | Injection volume (mL) |
|---|---|---|
| 150 | 1 | 1.2 |
| 225 | 2 | 1.8 (1.2 + 0.6) |
| 300 | 2 | 2.4 (1.2 + 1.2) |
| 375 | 3 | 3.0 (1.2 + 1.2 + 0.6) |

The dose of Omalizumab is based on both the patient's body weight and their total serum IgE level measured before treatment, according to the scheme shown in Table 3.

TABLE 3
Omalizumab doses, SQ Administration

| | Milligrams (mg) Per Dose | | | | | | |
|---|---|---|---|---|---|---|---|
| Baseline IgE | Body weight (kg) | | | | | | Frequency |
| (IU/mL) | 20-30 | >30-40 | >40-50 | >50-60 | >60-70 | >70-90 | of Dosing |
| >30-100 | 150 | 150 | 150 | 150 | 150 | 150 | Q4 wk |
| >100-200 | 150 | 150 | 300 | 300 | 300 | 300 | |
| >200-300 | 150 | 300 | 300 | 300 | 225 | 225 | Q2 wk |
| >300-400 | 300 | 300 | 225 | 225 | 225 | 300 | |
| >400-500 | 300 | 225 | 225 | 300 | 300 | 375 | |
| >500-600 | 300 | 225 | 300 | 300 | 375 | | Not Dosed |
| >600-700 | 225 | 225 | 300 | 375 | | | |
| >700-800 | 225 | 300 | 375 | | | | |
| >800-900 | 225 | 300 | 375 | | | | |
| >900-1000 | 300 | 375 | | | | | |
| >1000-1100 | 300 | 375 | | | | | |
| >1100-1200 | 300 | | | | | | |
| >1200-1300 | 375 | | | | | | |

The 2-weekly schedule (Q2wk) may be adopted if the dose will present too large a volume of injection for administration at one visit.

Dosing in SAR: For SAR, Omalizumab 300 mg may be administered subcutaneously every 3 or 4 weeks. This results in a fixed dose of 300 or 400 mg per 4 weeks. Dosing frequency is determined by baseline serum total IgE level (IU/mL) measured before the start of treatment (Table 4). In patients with known SAR Omalizumab therapy may be initiated 2 weeks prior to the anticipated start of the pollen season.

TABLE 4
Omalizumab doses for adults and children (6 years of age and older),
SQ Administration, Example for SAR

| Baseline Serum IgE (IU/mL) | mg per dose | Frequency of dosing |
|---|---|---|
| 30-150 | 300 | every 4 weeks |
| 150-700 | 300 | every 3 weeks |

All tests are conducted in accordance with GLP (Good Laboratory Practice) principles following procedures known in the art.

Various parameters of the study described above can be modified, e.g. in order to optimize the dosage for special diseases or indications mentioned herein, to cope with tolerability problems during the study or to obtain similar or identical results with less efforts. For example, a different subject population can be involved in such a clinical trial, the term of the placebo run-in period (period 1) can be changed, i.e. it can be extended, shortened or deleted; the visit schedule can be extended; the visit instructions can be changed; or one or more of the parameters to be determined during the study mentioned above can be deleted or the determination of additional parameters (see below) can be added.

Additional parameters can be determined in the course of the study, e.g. by additional tests. For example, a peak flow meter, a simple device to measure lung volume, can be used at home daily to check on lung functions. Peak flow values of 50-80% of an individual's personal best indicate a moderate asthma exacerbation, while values below 50% indicate a severe exacerbation.

If the COMBINATION OF THE INVENTION is contemplated as the combination of Omalizumab and 33-epichloro,33-desoxyascomycin (pimecrolimus), then 33-epichloro,33-desoxyascomycin (pimecrolimus) may be co-administered, either in the same vehicle or in separate vehicles. The compounds may be administered as a fixed combination or may be administered in separate dosage forms. The compounds may also be administered at substantially the same time, e.g. within fifteen minutes or less, either in the same vehicle or in separate vehicles. Alternatively, Omalizumab may be administered before administering 33-epichloro,33-desoxyascomycin (pimecrolimus), e.g. four or two weeks, or only three days, or as little as about 15 min before receiving 33-epichloro,33-desoxyascomycin (pimecrolimus). Patients already under treatment with Omalizumab may receive 33-epichloro,33-desoxyascomycin (pimecrolimus) at any time within the treatment intervals of Omalizumab, such as the two weeks or four weeks intervals as described in Example 1 and Table 3, for example.

Results

The combined administration of the COMBINATION OF THE INVENTION, such as in particular the combination of Omalizumab and 33-epichloro,33-desoxyascomycin (pimecrolimus), results in a beneficial, especially a synergistic, therapeutic effect, especially on allergic disease, and/or in additional benefits, or an improved safety profile, compared to a monotherapy applying only one of the active ingredients used in the COMBINATION OF THE INVENTION. Further benefits are, e.g., that lower doses of the individual drugs to be combined according to the present invention can be used to reduce the dosage, for example, that the dosages need not only often be smaller but are also applied less frequently, or can be used in order to diminish the incidence of side effects.

Furthermore, in a number of combinations as disclosed herein the side-effects observed with one of the active ingredients surprisingly do not accumulate on application of the COMBINATION OF THE INVENTION.

Additionally, the beneficial therapeutic effects, additional benefits and also the surprising beneficial effects are observed especially in patients poorly controlled by monotherapy with one of the components of the COMBINATION OF THE INVENTION.

### Example 2

Animal Studies

Animals

Male BALB/c mice (Harlan, UK), approximately 7 weeks old were used throughout the study. The mice were maintained on ovalbumin (OVA)-free diets. All experimental protocols were in accordance with the Home Office 1986 Animals Scientific Act and were approved by the NHRC animal welfare committee

Sensitization and Airway Challenge

BALB/c mice were immunized intraperitoneally with 0.2 ml 0.9 % wttvol NaCI (Saline) containing 100 μg of ovalbumin (5× crystallized, Sigma, UK) adsorbed in 1.6 mg aluminum hydroxide (Sigma). 14 days following the initial injection, mice were similarly boosted with the antigen/adjuvant. On Day 21, mice were challenged once with a 20 minute exposure to aerosolized ovalbumin, using a 20 mg/ml ovalbumin solution in phosphate-buffered saline. Control animals were similarly immunized with ovalbumin and challenged with PBS.

In Vivo Compound Treatment Schedule

Mice were administered anti-murine IgE antibody (1-5; Coyle A J, et al. (1996) J Exp Med; 183(3):1303-10) or rat IgG (Sigma) intravenously just prior to the sensitization boost. Mice received one single dose of 200 μ5g of antibody delivered in 50 μg via the tail vein. Mice received an oral administration of 33-epichloro,33-desoxyascomycin (pimecrolimus) 20% solid dispersion (10 or 30 mg/kg) or vehicle at 1 h prior to and 6 h post-aerosolized challenge.

Bronchoalveolar lavage (BAL) Cell Counts and Differentiation

At 24 h after the challenge, mice were anaesthetized by an intraperitoneal injection of 4 mg/kg sodium pentobarbital (Rhone Merieux, Harlow,UK). BAL fluid was collected by cannulating the trachea and washing the lungs with a total of 1.2 ml saline solution (3×0.4 ml each). Total cell count was determined and cytospin preparation (Shandon Scientific Ltd., Cheshire, UK) performed. Cells were stained with Diff-Quik (Baxter Dade AG, Dudingen, Switzerland) and a differential count of 200 cells performed using standard morphological criteria. Results are expressed as absolute counts of differential and total cell counts in the BAL.

Results are expressed as means t SEM of the indicated number of animals (Table 5). One way analysis of variance (ANOVA) was used to determine significance among the groups. If a significant variance was found, Student's t test was used to assess comparability between means. A value of P<0.05 was considered significant.

Results

Aerosolization of ovalbumin induced an increase in eosinophil and macrophage numbers in the BAL fluid at 24 h compared to PBS-aerosolized controls. Administration of anti-murine IgE antibody at 10 mg/kg given at time of antigen boost, no significant effect on airway inflammation was observed in this animal model. Furthermore, oral administration of 10 mg/kg 33-epichloro,33-desoxyascomycin (pimecrolimus) alone had no significant effect on OVA-induced inflammation in the BAL fluid, although increasing the dose to 30 mg/kg reduced the airway eosinophilia by 78%. However, administration of 10 mg/kg 33-epichloro,33-desoxyascomycin (pimecrolimus) in mice pre-administered anti-murine IgE Ab (10 mg/kg), reduced the eosinophilia by 66%. This results suggests that such combination therapy confers additional protection against allergen-induced allergic airway inflammation, compared to administration of either reagent alone, at these doses.

TABLE 5
Summary of differential leukocyte counts in BAL (×10⁵/ml)

| Treatment | | | | | | Total cell |
|---|---|---|---|---|---|---|
| Group | Challenge | Neutrophils | Eosinophils | Macrophages | Lymphocytes | counts |
| Isotype/vehicle | PBS | 0.01 ± 0.01 | 0.00 | 0.48 ± 0.07 | 0.00 | 0.50 ± 0.07 |
| Isotype/vehicle | OVA | 0.08 ± 0.02 | 0.66 ± 0.10 | 1.43 ± 0.20 | 0.00 | 2.00 ± 0.24 |
| Anti-IgE/vehicle | OVA | 0.13 ± 0.04 | 0.92 ± 0.20 | 0.86 ± 0.12 | 0.00 | 1.92 ± 0.30 |
| Isotype/ | OVA | 0.08 ± 0.04 | 0.65 ± 0.26 | 0.93 ± 0.15 | 0.00 | 1.63 ± 0.40 |
| Ascomycin* | | | | | | |
| 10 mg/kg | | | | | | |
| Anti-IgE/ | OVA | 0.06 ± 0.03 | 0.22 ± 0.08 | 0.67 ± 0.10 | 0.00 | 0.95 ± 0.15 |
| Ascomycin* | | | | | | |
| 10 mg/kg | | | | | | |
| Isotype/ | OVA | 0.03 ± 0.01 | 0.15 ± 0.04 | 0.85 ± 0.18 | 0.00 | 1.03 ± 0.22 |
| Ascomycin* | | | | | | |
| 30 mg/kg | | | | | | |
| Anti-IgE/ | OVA | 0.01 ± 0.01 | 0.05 ± 0.02 | 0.66 ± 0.18 | 0.00 | 0.72 ± 0.27 |
| Ascomycin* | | | | | | |
| 30 mg/kg | | | | | | |

*Ascomycin is 33-epichloro, 33-desoxyascomycin (pimecrolimus)

1. A pharmaceutical composition comprising as active ingredients an anti-IgE antibody and at least one further antiallergic compound selected from the group consisting of anti-inflammatory agents, leukotriene modifiers, bronchodilators, antihistamines, interleukin antagonists, mast cell inhibitors, and Immunotherapeutical agents, in which the active ingredients are present in free form or in the form of a pharmaceutically acceptable salt and optionally at least one pharmaceutically acceptable carrier; for simultaneous, separate or sequential use.

2. The composition according to claim 1 wherein the anti-IgE antibody and the at least one further antiallergic compound are comprised in a single pharmaceutical formulation.

3. The composition according to claims 1 wherein the anti-IgE antibody is Omalizumab.

4. The composition according to claim 3, characterized in that Omalizumab is administered in a concentration dependent on the baseline IgE level of the patient to be treated.

5. The composition according to claim 1, characterized in that the combination comprises a further monoclonal antibody.

6. The composition according to any one of claims 1, characterized in that the composition comprises at least one immunotherapeutical agent as the further pharmaceutically active compound.

7. The composition according to claim 6, characterized in that the immunotherapeutical agent is a macrolide T-cell immunomodulator or immunosuppressant.

8. The composition according to claim 7, characterized in that the macrolide T-cell immunomodulator or immunosuppressant is a FKBP12-binding calcineurin inhibitor or mitogen-activated kinase modulator or inhibitor.

9. The composition according to claim 8, characterized in that the FKBP12-binding calcineurin inhibitor or mitogen-activated kinase modulator or inhibitor is an asco- or rapamycin.

10. The composition according to claim 9, characterized in that the ascomycin is 33-ephichloro, 33-desoxyascomycin (pimecrolimus).

11. The composition according to claim, wherein the anti-IgE antibody is Omalizumab and the immunotherapeutical agent is 33-epichloro,33-desoxyascomycin (pimecrolimus).

12. The composition according to claim 1, wherein the anti-IgE antibody and antiallergic compound are present in respective amounts which are jointly therapeutically effective against allergic disease.

13. The composition according to claim 12 wherein the anti-IgE antibody and antiallergic compound are present in respective amounts which are jointly therapeutically effective against asthma.

14. A method for the prevention, delay of progression or treatment of allergic disease comprising administering to a warm-blooded animal a therapeutically effective amount of a composition according to claim 1.

15. A method of treatment of allergic disease comprising administering to a warm-blooded animal a therapeutically effective amount of an anti-IgE antibody and an antiallergic compound.

16. A method of treatment of allergic disease according to claim 15, wherein the anti-IgE antibody is Omalizumab.

17. A method of treatment of allergic disease according to claim 15, wherein the antiallergic compound comprises an immunotherapeutical agent.

18. A method of treatment of allergic disease according to claim 17, wherein the immunotherapeutical agent is a macrolide T-cell immunomodulator or immunosuppressant.

19. A method of treatment of allergic disease according to claim 18 wherein the macrolide T-cell immunomodulator or immunosuppressant is a FKBP12-binding calcineurin inhibitor or mitogen-activated kinase modulator in inhibitor.

20. A method of treatment of allergic disease according to claim 19 wherein the FKBP12-binding calcineurin inhibitor or mitogen-activated kinase modulator or inhibitor is an asco- or rapamycin.

21. A method of treatment of allergic disease according to claim 20 wherein the ascomycin is 33-epichloro,33-desoxyascomycin (pimecrolimus).

22. A method of treatment of allergic disease according to claim 15 wherein the anti-IgE antibody is Omalizumab and the immunotherapeutical agent is 33-epichloro,33-desoxyascomycin (pimecrolimus).

23. A method of treatment of allergic disease wherein an anti-IgE antibody and an antiallergic compound are co-administered.

24. A method of treatment of allergic disease wherein Omalizumab and 33-epichloro,33-desoxyascomycin (pimecrolimus) are co-administered.

25. A method of treatment of allergic disease according to claim 23 wherein Omalizumab is administered before 33-epichloro,33-desoxyascomycin (pimecrolimus).

26. The use of a composition according to claim 1 in medicine.

27. The use of a composition according to claim 1 for the manufacture of a medicament for the treatment of a warm-blooded animal having allergic disease.

28. Use of the composition according to claim 1 for the prevention or treatment of allergic disease.

29. The use of Omalizumab and 33-epichloro,33-desoxyascomycin (pimecrolimus) for the manufacture of a medicament for the treatment of a warm-blooded animal having allergic disease.

30. The use of an antiallergic compound for the manufacture of a medicament for the treatment of a patient under treatment with Omalizumab.

31. The use of an antiallergic compound according to claim 30, wherein the antiallergic compound is an immunotherapeutical agent.

32. The use of an antiallergic compound according to claim 31, wherein the immunotherapeutical agent is a macrolide T-cell immunomodulator or immunosuppressant.

33. The use of an antiallergic compound according to claim 32, wherein the macrolide T-cell immunomodulator or immunosuppressant is a FKBP12-binding calcineurin inhibitor or mitogen-activated kinase modulator or inhibitor.

34. The use of an antiallergic compound according to claim 33,wherein the FKBP12-binding calcineurin inhibitor or mitogen-actived kinase modulator or inhibitor is an asco- or rapamycin.

35. The use of an immunotherapeutical agent according to claim 34, wherein the ascomycin is 33-epichloro,33-desoxyascomycin (pimecrolimus).

36. A kit comprising as active agent a composition according to claim 1 together with instructions for simultaneous, separate or sequential use thereof in the prevention, delay of progression or treatment of allergic disease.
